(19) United States
(12) Patent Application Publication
(10) Pub. No.: US 2018/0200398 A1
(43) Pub. Date: Jul. 19, 2018

(54) DEODORIZING STORAGE CONTAINER

(71) Applicant: Steven Owen Wigley, Toano, VA (US)

(72) Inventor: Steven Owen Wigley, Toano, VA (US)

(21) Appl. No.: 15/839,857

(22) Filed: Dec. 13, 2017

Related U.S. Application Data
(60) Provisional application No. 62/447,902, filed on Jan. 18, 2017.

(51) Int. Cl. A61L 9/00; D06F 95/00; B01D 46/00

(52) U.S. Cl. CPC A61L 9/00; D06F 95/002; A61L 2209/22; B01D 46/0043; A61L 2209/14; B01D 46/0038

(57) ABSTRACT

A deodorizing storage container includes a substantially hollow body, a rack, a lid, a plurality of air intake holes and an air-purification system. The substantially hollow body is defined by an inner chamber, an interior wall and an exterior wall. The rack may be removably positioned within the inner chamber of the substantially hollow body. The lid may be removably attached to the substantially hollow body and the plurality of air intake holes may be located at a bottom portion of the substantially hollow body. The deodorizing storage container provides users with a deodorizing storage container capable of containing and effectively eradicating undesirable odors due to smelly shoes, socks, clothes, jerseys, uniforms, pads, sporting equipment and much more. It allows athletes, parents, college students, outdoorsmen and others to be in close quarters with their stored smelly equipment that would otherwise contaminate the air quality of the inhabited space.

### CROSS REFERENCE TO RELATED APPLICATION

The present application is related to and claims priority to U.S. Provisional Patent Application No. 62/447,902 filed Jan. 18, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of storage containers and more specifically relates to a deodorizing storage container.

### 2. Description of Related Art

After a workout or participation in strenuous activities, clothing and equipment radiate unwanted odors. Even casual clothing during casual use, such as a pair of sneakers, will begin to emit unwanted odors after frequent use. With used clothing and equipment typically being stored in a closet or laundry basket and close to inhabitants, odors regularly and relentlessly permeate the air. People are forced to tolerate these odors until the clothing is washed or thrown out or equipment is cleaned, either way the smells contaminate the room and are dispersed throughout the house. The garment owners may use air fresheners, candles and other deodorizers to combat the smell, however, these methods are typically ineffective as they mask odors and can make them worse. They certainly do not confine and treat the odor at the source.

Many people use deodorizers to control odors. Odors can be caused by a number of factors, and are generally unwanted and unpleasant. An unpleasant odor can quickly fill up and overcome a small space; removing the source of the odor can alleviate it, or the odor may be masked by a more pleasant scent. A suitable solution is desired.

U.S. Pub. No. 2008/0118411 to Ralph D'Arinzo relates to an ozone deodorizing and disinfecting portable containers. The described ozone deodorizing and disinfecting portable containers include a portable storage apparatus for disinfecting, deodorizing and drying objects. The device for the apparatus comprising: a bag having at least one interior chamber formed by at least one outer wall for containing objects therein, ozone generator means for creating ozone, air distribution means for the movement and circulation of ozonated air within, throughout and out of the at least one interior chamber. The ozone generation means and air circulation means are located within or in direct circulatory contact with the one interior chamber of the bag to force ozonated air into and throughout the one interior chamber of the bag, and means on the outer wall permitting expulsion of air from within the interior chamber of the bag to the environment outside the bag. Resulting in the disinfection, deodorization and drying of objects placed within the apparatus through contact with the ozone and air distributed therein.

### BRIEF SUMMARY OF THE INVENTION

In view of the foregoing disadvantages inherent in the known storage container art, the present disclosure provides a novel deodorizing storage container. The general purpose of the present disclosure, which will be described subsequently in greater detail, is to provide a deodorizing storage container capable of effectively eradicating undesirable odors due to smelly shoes, socks, jerseys, uniforms, athletic equipment and much more. It allows athletes, parents, college students, outdoorsmen and others to be in close quarters with their stored smelly equipment that would otherwise contaminate the air quality of the inhabited space.

A deodorizing storage container is disclosed herein. The deodorizing storage container includes a substantially hollow body, a rack, a lid, a plurality of air intake holes, an air-purification system with fan, activated carbon filter and an on/off switch. The substantially hollow body is defined by an inner chamber, an interior wall and an exterior wall. The rack may be removably positioned within the inner chamber of the substantially hollow body. The lid may be removably attached to the substantially hollow body and the plurality of air intake holes may be located at a bottom portion of the substantially hollow body. The deodorizing storage container is configured to confine, contain and reduce undesirable odors contained within.

A method of using deodorizing storage container is also disclosed herein. The method of using deodorizing storage container may comprise the steps of: providing a deodorizing storage container; providing power to the deodorizing air purification system having a fan and carbon filter combination attached to the storage container; drawing outside air into the deodorizing storage container; circulating air inside the deodorizing storage container; and discharging the inside air from the deodorizing storage container through the air-purification system's activated-carbon-based air-deodorizing filter.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a deodorizing storage container, constructed and operative according to the teachings of the present disclosure.

FIG. 1 is a perspective view of the deodorizing storage container during a pre ‘in-use’ condition, according to an embodiment of the disclosure.

FIG. 2A is a perspective view of the deodorizing storage container of FIG. 1, during an ‘in-use’ condition drawing in fresh air, according to an embodiment of the present disclosure.

FIG. 2B is a perspective view of the deodorizing storage container of FIG. 1, during an ‘in-use’ condition circulating air within the container thereby capturing the foul air, according to an embodiment of the present disclosure.

FIG. 2C is a perspective view of the deodorizing storage container of FIG. 1, during an ‘in-use’ condition drawing the foul air to the deodorizing fan/filter combination and discharging the clean filtered air to the adjacent environment, according to an embodiment of the present disclosure.

FIG. 3 is a perspective view of the deodorizing storage container of FIG. 1, during an ‘in-use’ condition in what are close quarters to the inhabitants of a vehicle, according to an embodiment of the present disclosure.

FIG. 4 is a perspective view of the deodorizing storage container of FIG. 1, during an ‘in-use’ condition configured as a laundry hamper, according to an embodiment of the present disclosure.

FIG. 5 is an exploded perspective of the air-purification system mounted on the exterior wall of the deodorizing storage container of FIG. 1.

FIG. 6 is a flow diagram illustrating a method of use for deodorizing storage container, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a storage container and more particularly to a deodorizing storage container as used to contain odors and improve the air quality by not permitting contained odors to emanate throughout the adjacent space.

Generally, the present invention provides users with a deodorizing storage container capable of effectively eradicating undesirable odors due to smelly shoes, socks, jerseys, uniforms, athletic equipment, and much more. It allows athletes, parents, college students, outdoorsmen and others to be in close quarters with their stored smelly equipment that would otherwise contaminate the air quality of the inhabited space. It features an air-purification system containing a fan, an activated carbon based deodorizing air filter and fan guards. The air is drawn in by the fan, circulates around contents gathering odors and is discharged from the device through the air-purification system, effectively removing the odors. This enables parents to transport aspiring athletes and their used sporting equipment, such as cleats, socks, pads, uniforms and the like without the pungent smells engulfing the confined space of a vehicle or room. The present invention, offered in an alternative version functioning as a laundry hamper with a removable mesh bag, allows users to easily store and keep the smell of used dirty laundry from overwhelming a room's atmosphere prior to cleaning or washing.

The deodorizing storage container is an odor reducing device used for sweaty, smelly clothing, athletic garments and equipment immediately after use and during periods of storage. Foul air being contained and treated within the deodorizing device is not permitted to circulate and contaminate outside air. The device is comprised of a storage container with integrated air-purification system. Air intake holes, a rack, and a lid with an airtight seal, hinges and latching device, support the system. A fan and an activated carbon based air deodorizing filter is installed on the container and is the mechanism that provides the odor reducing process. The fan is sized and positioned to create a negative pressure inside the container allowing air to be pulled into the deodorizing storage container, circulated around the garments gathering odors, and blown out through the deodorizing activated carbon based filter. In this way, odors are effectively removed from the air within the deodorizing storage container and without fouling the surrounding atmosphere.

An alternate version of the deodorizing storage container may be available in the form of a laundry hamper with a breathable mesh bag in lieu of a rack. The hamper works in the same manner, though it is larger and taller to permit more volume for storage prior to regular cleaning. The deodorizing storage container can be constructed using plastic and/or other suitable materials. A breathable mesh bag can be constructed of breathable cloth fabric, netting, plastic, pliable wire netting and/or other suitable materials and has a closing device such as a draw string, zipper or other suitable closing mechanism to permit transport when removed for regular cleaning as does a duffle bag. The lid features self-closing hinges, a neoprene seal and latching device to ensure airtight securement. The lid can further be configured to open with the step of one's foot onto a lid opening mechanism that is integral to the laundry hamper.

The rack can be constructed of plastic, metal and/or other suitable material. The rack effectively holds contents off of the bottom and sides of the container which allows air to circulate around the entire garment emanating odors. The mesh bag, in the laundry hamper configuration, is positioned in such a way as to hold the contents off of the bottom and sides of the deodorizing storage container which allows air to circulate around the entire garment emanating odors. The rack maybe held in place by legs integral to the manufactured rack or by brackets secured to the sides of the container or by braces molded into the sides of the container. The mesh bag maybe held in place by brackets secured to the sides of the container or by braces molded into the sides of the container or by other suitable means.

The deodorizing storage container can be available in medium (M), large (L), extra-large (XL), and extra-extra-large (XXL) sizes in order to accommodate different types and sizes of clothing and/or athletic equipment.

The air-purification system's fan is preferably powered by 12V AC/DC adapters permitting transport and use in vehicles. The air-purification system's filter maybe an activated carbon based filter which is removable to permit cleaning and/or replacement. Both the fan and filter are engineered and sized to accommodate the size and volume of the deodorizing storage container and the air flow required to ensure a negative air pressure is achieved inside the deodorizing storage container during the ‘in-use’ condition.

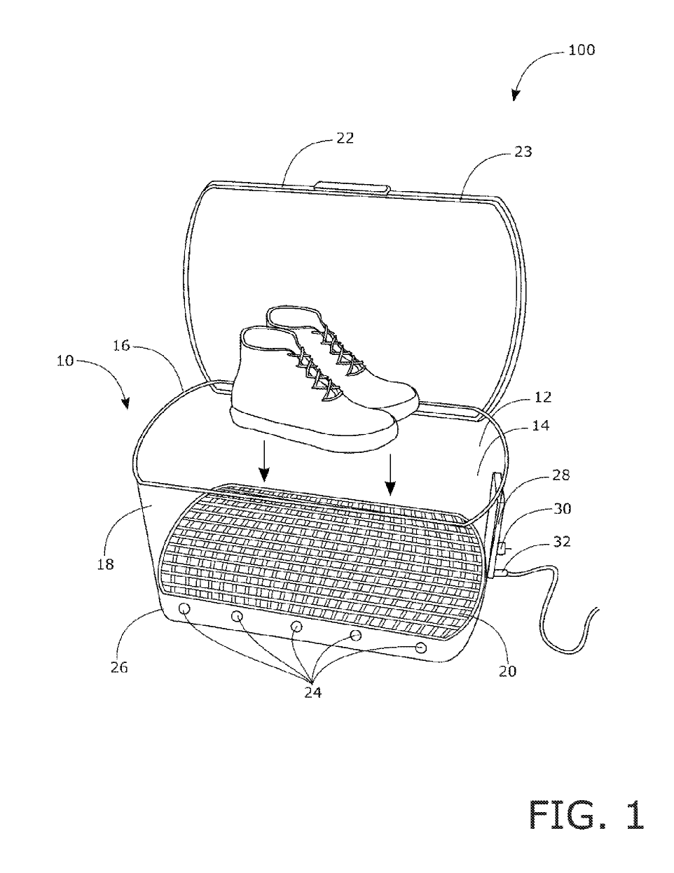

Referring now more specifically to the drawings by numerals of reference, there is shown in FIGS. 1-6, various views of a deodorizing storage container 10. FIG. 1 shows a deodorizing storage container 10 at a pre ‘in-use’ condition 100, according to an embodiment of the present disclosure. As illustrated, the deodorizing storage container 10 which may include a substantially hollow body 12, a rack 20, a lid 22 with airtight seal 23, a plurality of air intake holes 24 and the air-purification system 28. The substantially hollow body 12 is defined by an inner chamber 14, an interior wall 16 and an exterior wall 18. The rack 20 may be removably positioned within the inner chamber 14 of the substantially hollow body 12 so as to hold contents off of the bottom and sides of the container. The lid 22 may be removably attached to the substantially hollow body 12 and the plurality of air intake holes 24 may be located at a bottom portion 26 of the substantially hollow body 12 below the rack 20. The air-purification system 28 is mounted in an opening in the interior wall 16 and exterior wall 18 of the container above the rack 20 and may be operated by an on/off switch 30 that is powered by 12V AC/DC power adapter 32. The deodorizing storage container 10 is configured for reducing undesirable odors.

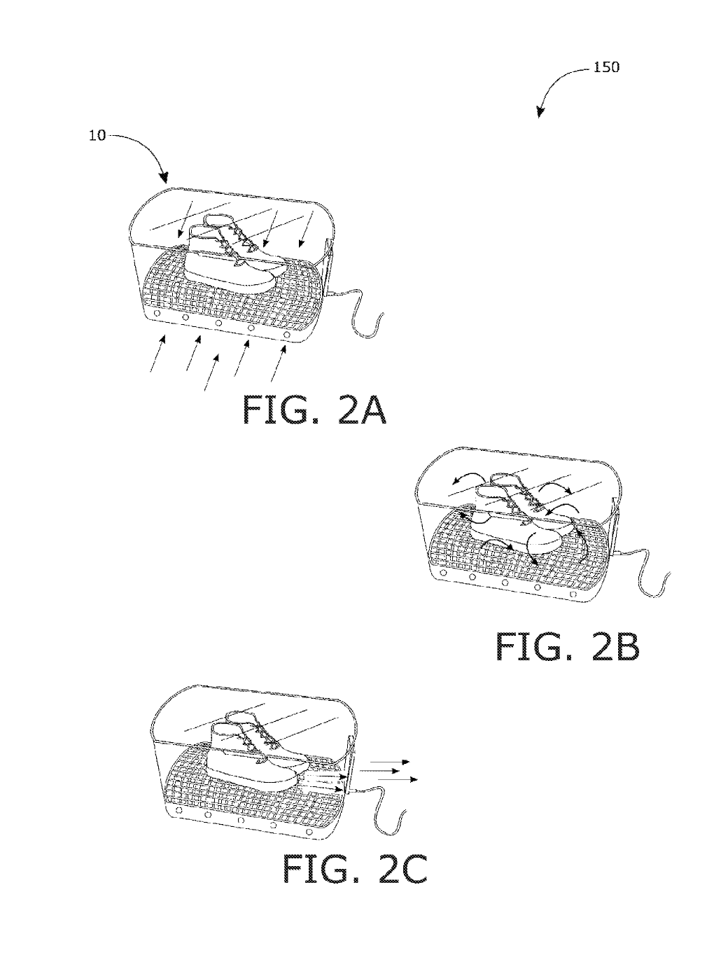

FIG. 2A-2C show perspective views of the deodorizing storage container 10 of FIG. 1 during an ‘in-use’ condition 150, according to an embodiment of the present disclosure. As above, the deodorizing storage container 10 may include the substantially hollow body 12, the rack 20, the lid 22, the plurality of air intake holes 24 and air-purification system 28.

FIG. 2A the lid 22 is in a closed and latched position. The plurality of air intake holes 24 are configured to allow outside air to be freely drawn into the deodorizing storage container 10. In a preferred embodiment, the plurality of air intake holes 24 includes an arrangement with an appropriate number of intake holes 24, and of appropriate size, to permit a negative air pressure within the storage container, due to the flow of air pulled by the fan of the air-purification system 28 being engineered to be greater than the flow of air permitted to be drawn into the intake holes 24. In this way the inner chamber is maintained at a pressure level below external ambient (i.e. less air supplied than exhausted) to help prevent exfiltration. The plurality of air intake holes 24 is in fluid communication with the inner chamber 14 and fan of the air-purification system 28 of the deodorizing storage container 10. The bottom portion of the substantially hollow body 26 is located below the rack 20 and is the location for the intake holes 24.

FIG. 2B shows that this air pressure arrangement between the intake holes 24 and fan ensures that the internal air that has become contaminated with odor is not permitted to exfiltrate or escape out through the intake holes 24 or elsewhere. FIG. 2A air drawn in must circulate within storage container due to the pressure differential between the inside air and outside air. In other embodiments, additional air intake holes 24 may be provided for suitable air flow. The air-purification system 28 is located on the exterior wall 18 of the deodorizing storage container 10 and above the rack 20. FIG. 2C shows the contaminated air being drawn towards the fan of the air-purification system 28 and discharged out through the activated carbon filter of the air-purification system 28 and into the surrounding environment. In other embodiments, the deodorizing storage container 10 may be a laundry hamper.

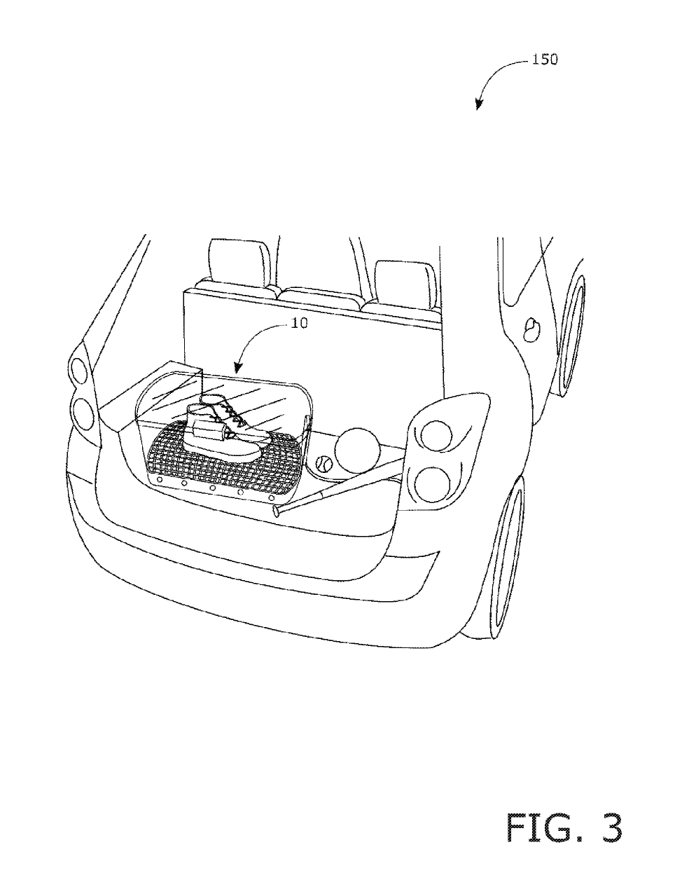

FIG. 3 shows a perspective view of the deodorizing storage container 10 of FIG. 1, during an ‘in-use’ condition 150, according to an embodiment of the present disclosure. As above, the deodorizing storage container 10 may include the substantially hollow body 12, the rack 20, the lid 22, the plurality of air intake holes 24 and air-purification system 28. The hollow body 12 may be 3D-rectangular in shape. The rack 20 is configured to hold malodorous athletic garments and malodorous athletic equipment such as shoes, socks, clothes, jerseys, cleats, pads, and uniforms off of the bottom and interior walls 16 of the container. The lid 22 may be removably attached to the substantially hollow body 12 and the plurality of air intake holes 24 may be located at a bottom portion of the substantially hollow body 12. The lid 22 is configured to provide an airtight seal 23 and maybe hinged and closed with a latching device or other suitable fastening means. The airtight seal 23 may include a neoprene seal, gasket or seal of other suitable material, and ensures an airtight container when lid is in the closed position. The deodorizing storage container 10 further includes an air-purification system 28 including a fan, an activated carbon-based air-deodorizing filter and can be powered by 12V AC/DC adapters to permit transportability.

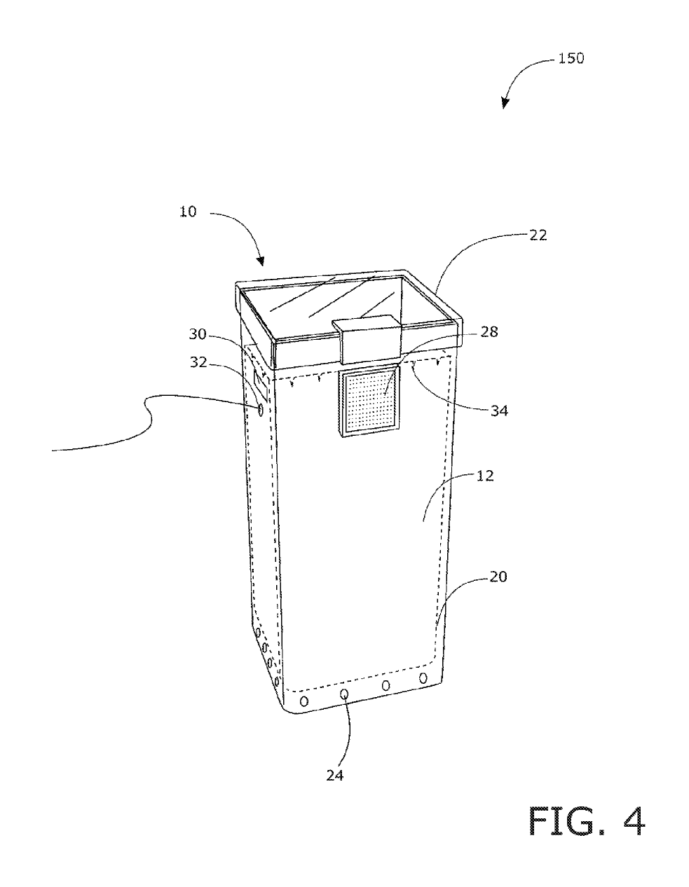

FIG. 4 shows a perspective view of the deodorizing storage container 10 of FIG. 1, during an ‘in-use’ condition 150, according to an embodiment of the present disclosure. In this FIG. 4 embodiment, the deodorizing storage container 10 is configured as a laundry hamper. As above, the deodorizing storage container 10 may include the substantially hollow body 12, the rack 20, the lid 22, the plurality of air intake holes 24 and the air-purification system 28. The deodorizing storage container 10 is configured for reducing undesirable odors. The undesirable odors may be produced from malodorous athletic garments, used clothing and malodorous athletic equipment. The air-purification system 28 may be operated by a power source including an on/off switch 30 and 12V AC/DC adapters 32 to allow for the transportability of the deodorizing storage container 10. In this FIG. 4 embodiment, the rack 20 is configured as a mesh bag held in place by brackets 34 secured to the interior walls of the container.

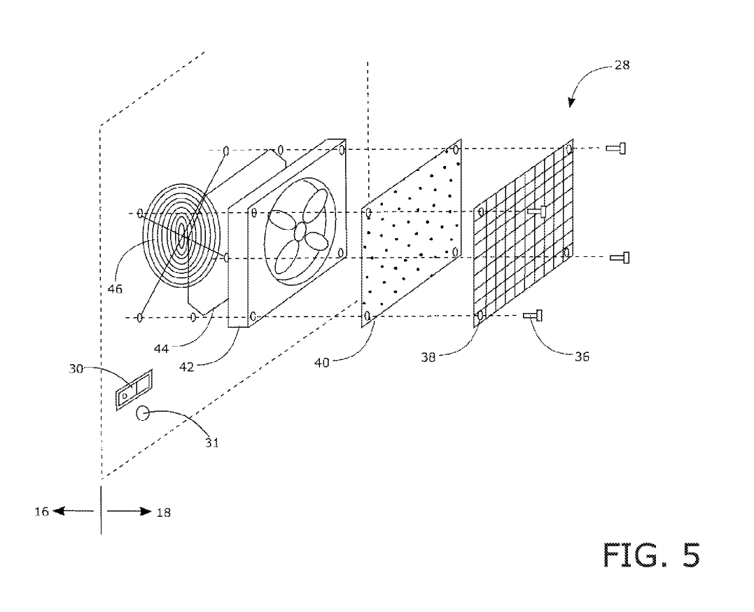

FIG. 5 shows an exploded view of an air-purification system 28 of FIG. 1, according to an embodiment of the present disclosure. The air-purification system 28 may be operated by an on/off switch 30 that controls the power source that may include a power jack 31 for 12V AC/DC adapters to allow transportability of the deodorizing storage container. This air-purification system 28 is positioned in a cutout or opening 44 in the interior wall 16 and exterior wall 18 and may contain a fan guard 46, the fan 42, the activated carbon filter 40 and wire mesh filter/fan guard 38. The activated carbon filter 40 is a removable element for cleaning or replacement after periods of prolonged operation of the deodorizing storage container 10 and can be secured in place by fasteners 36.

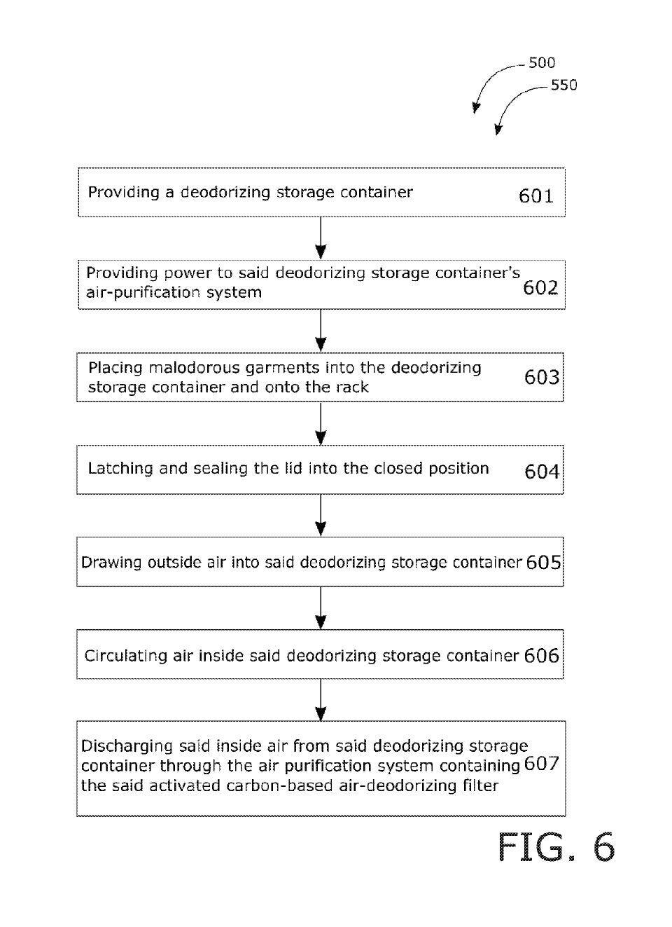

FIG. 6 is a flow diagram 550 illustrating a method of reducing undesirable odors 500, according to an embodiment of the present disclosure. As illustrated, the method of reducing undesirable odors 500 may include the steps of: step one, providing a deodorizing storage container 10; step two, providing power to the air-purification system 28; step three, placing malodorous garments or equipment into the deodorizing storage container 10 and onto the rack 20; step four, latching and sealing the lid 22 into the closed position; step five, drawing outside air into the deodorizing storage container 10; step six, circulating air inside the deodorizing storage container 10; and step seven, discharging the inside air from the deodorizing storage container 10 through the air-purification system 28 containing the activated carbon-based air-deodorizing filter.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as engineering, design preference, user preferences, marketing preferences, cost, structural requirements, available materials, technological advances, etc., other methods for deodorizing storage container 10 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A deodorizing storage container for reducing undesirable odors, said deodorizing storage container comprising:
a substantially hollow body, said substantially hollow body having an inner chamber, an interior wall and an exterior wall;
a rack, said rack being removably positioned within said inner chamber of said substantially hollow body;
a lid, said lid, containing a seal, being removably attached to said substantially hollow body;
a plurality of air intake holes, said plurality of air intake holes is located at a bottom portion of said substantially hollow body;
an air-purification system, being attached to the substantially hollow body; and
wherein said deodorizing storage container is configured for reducing undesirable odors.

2. The deodorizing storage container of claim 1, wherein said hollow body is 3D-rectangular in shape.

3. The deodorizing storage container of claim 1, wherein said rack is configured to hold malodorous athletic garments and malodorous athletic equipment.

4. The deodorizing storage container of claim 3, wherein said malodorous athletic garments and malodorous athletic equipment is selected from the group consisting of shoes, socks, clothes, jerseys, cleats, pads, uniforms and equipment.

5. The deodorizing storage container of claim 1, wherein said lid is configured to provide an airtight seal.

6. The deodorizing storage container of claim 1, further comprising an air-purification system.

7. The deodorizing storage container of claim 6, wherein said air-purification system includes a fan.

8. The deodorizing storage container of claim 7, wherein said air-purification system further includes an activated carbon-based air-deodorizing filter.

9. The deodorizing storage container of claim 1, wherein said plurality of air intake holes is configured to allow outside air to flow freely into said deodorizing storage container.

10. The deodorizing storage container of claim 1, wherein said plurality of air intake holes includes at least 1 air intake hole(s).

11. The deodorizing storage container of claim 1, wherein said bottom portion of said substantially hollow body is located below said rack.

12. The deodorizing storage container of claim 1, wherein said plurality of air intake holes is in fluid communication with said inner chamber of said deodorizing storage container.

13. The deodorizing storage container of claim 6, wherein said air-purification system is located on said exterior wall of said deodorizing storage container and in fluid communication with the interior chamber and exterior environment.

14. The deodorizing storage container of claim 6, wherein said air-purification system is operated by a power source of said deodorizing storage container.

15. The deodorizing storage container of claim 6, wherein said air-purification system includes a power jack for a power source to include 12V AC/DC adapters to allow transportability of said deodorizing storage container.

16. The deodorizing storage container of claim 5, wherein said airtight seal includes a neoprene seal, hinges and latching device.

17. A deodorizing storage container for reducing undesirable odors, said deodorizing storage container comprising:
a substantially hollow body, said substantially hollow body having an inner chamber, an interior wall and an exterior wall;
a rack, said rack being removably positioned within said inner chamber of said substantially hollow body;
a lid, said lid being removably attached to said substantially hollow body;
a plurality of air intake holes, said plurality of air intake holes is located at a bottom portion of said substantially hollow body;
an air-purification system, said air-purification system is located in an opening in the interior and exterior wall;
wherein said deodorizing storage container is configured for reducing undesirable odors;
wherein said hollow body is rectangular in shape;
wherein said rack is configured to hold malodorous athletic garments and equipment;
wherein said lid is configured to provide a substantially airtight seal;
further comprising an air-purification system;
wherein said air-purification system includes a fan;
wherein said air-purification system further includes an activated carbon-based air-deodorizing filter;
wherein said plurality of air intake holes is configured to allow outside air to be freely drawn into said deodorizing storage container;
wherein said plurality of air intake holes includes at least 1 air intake hole(s);
wherein said bottom portion of said substantially hollow body is located below said rack;
wherein said plurality of air intake holes is in fluid communication with said inner chamber of said deodorizing storage container;
wherein said air-purification system is located on said exterior wall of said deodorizing storage container and in fluid communication with the inner chamber and exterior environment;
wherein said air-purification system is operated by a power source including a power jack for 12V AC/DC adapters for allowing transportability of said deodorizing storage container; and
wherein said airtight seal includes a neoprene seal, hinges and latching device.

18. A method of reducing undesirable odors, said method comprising the steps of:
providing a deodorizing storage container, said deodorizing storage container having an integrated air-purification system, wherein said integrated air-purification system includes a fan and an activated carbon-based air-deodorizing filter;
providing power to said deodorizing storage container to turn said fan on;
placing malodorous garments and malodorous equipment into the deodorizing storage container and onto the rack;
securely closing the lid of the deodorizing storage container;
drawing outside air into said deodorizing storage container;
circulating air inside said deodorizing storage container; and
discharging said inside air from said deodorizing storage container through said carbon-based air-deodorizing filter.

19. The method of claim 18, wherein said undesirable odors is produced from malodorous athletic garments and malodorous athletic equipment selected from the group consisting of shoes, socks, clothes, jerseys, cleats, pads, and uniforms.

20. The method of claim 18, wherein the deodorizing storage container is a laundry hamper.
